(19) United States
(12) Patent Application Publication
(10) Pub. No.: US 2022/0370517 A1
(43) Pub. Date: Nov. 24, 2022

(54) METHODS OF TREATING ATOPIC DERMATITIS

(71) Applicants: Ichilov Tech Ltd., Tel-Aviv (IL); Yeda Research and Development Co. Ltd., Rehovot (IL)

(72) Inventors: Eran SEGAL, Ramat-HaSharon (IL); Jacob MASHIAH, Tel-Aviv (IL); Nitsan MAHARSHAK, Herzliya (IL)

(73) Assignees: Ichilov Tech Ltd., Tel-Aviv (IL); Yeda Research and Development Co. Ltd., Rehovot (IL)

(21) Appl. No.: 17/854,034

(22) Filed: Jun. 30, 2022

Related U.S. Application Data
(63) Continuation of application No. PCT/IL2020/051348, filed on Dec. 29, 2020.

(30) Foreign Application Priority Data
Dec. 31, 2019 (IL) 271778

(51) Int. Cl. A61K 35/741; A61K 35/745; A61K 35/742; A61K 35/24; A61P 17/00

(52) U.S. Cl. CPC A61K 35/741; A61K 35/745; A61K 35/742; A61K 35/24; A61P 17/00

(57) ABSTRACT

A method of treating atopic dermatitis (AD) in a subject in need thereof is provided. The method comprising administering to the subject a therapeutically effective amount of at least two species of bacteria.

### RELATED APPLICATIONS

This application is a Continuation of PCT Patent Application No. PCT/IL2020/051348, having international filing date of Dec. 29, 2020 which claims the benefit of priority of Israeli Patent Application No. 271778 filed on Dec. 31, 2019. The contents of the above applications are all incorporated by reference as if fully set forth herein in their entirety.

### FIELD AND BACKGROUND OF THE INVENTION

The present invention, in some embodiments thereof, relates to treatment of atopic dermatitis.

Atopic dermatitis (AD), a remitting-relapsing chronic eczematous pruritic skin disease, is one of the most common inflammatory skin diseases, affecting up to 20% of children and up to 10% of adults¹. The etiology of AD is multifactorial comprising of genetic predisposition, immune dysregulation and immune deviation mainly towards T_{H}2/T_{H}22 with some T_{H}1 and T_{H}17 overexpression, defective skin barrier function, abnormal microbial colonization, as well as environmental factors. Nearly 20% of all cases are considered as moderate or severe, affecting quality of life, and causing psychological, social, as well as financial burdens².

Until recently, the therapeutic ladder of atopic dermatitis consisted of dry skin care with moisturizers and topical treatments (mainly topical corticosteroids an calcineurin inhibitors), phototherapy, and immunosuppressant agents (such as cyclosporine A or methotrexate)³. Dupilumab, a fully human, monoclonal antibody inhibiting signaling of both IL-4 and IL-13 is the first and only targeted biologic treatment approved for moderate to severe AD. Other monoclonal antibodies as well as small molecules (Janus kinase inhibitors) targeting cytokines and intercellular signaling pathways downstream to cytokine receptors involved in AD² are under investigation.

Several studies address the role of microbiota in AD, with much emphasis on the skin microbiota, in particular Staphylococcus aureus^{4,5}. However, recent evidence supports the importance of the gut-skin axis, probably through the immune regulatory and immune activation capabilities of gut microbial antigens and metabolites⁶. According to the hygiene hypothesis there is an inverse relationship between AD and an early exposure to microbial agents⁶. Gastroenteritis during infancy as well as exposure to antibiotics during the first year of life are associated with AD in children^{7,8}. Indeed, gut microbial dysbiosis has been demonstrated in AD patients⁹. Moreover, probiotics have a positive effect on AD severity, alter the gut microbiota, and may propagate induction of regulatory T cells^{10,11.}

Additional background art includes:

U.S. Patent Application No. 20160317653;

Zeng et al. Journal of Immunology Research Volume 2019, Article ID 1603758.

Craig et al. Vet Med Sci. 2016 May; 2(2): 95-105.

### SUMMARY OF THE INVENTION

According to an aspect of some embodiments of the present invention there is provided a method of treating atopic dermatitis (AD) in a subject in need thereof comprising administering to the subject a therapeutically effective amount of at least two species of bacteria, wherein the at least two species of the bacteria are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, thereby treating the atopic dermatitis, wherein the bacteria are not comprised in a faecal microbiota transplantation (FMT).

According to an aspect of some embodiments of the present invention there is provided a therapeutically effective amount of at least two species of bacteria, wherein the at least two species of the bacteria are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594. SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, for use in the treatment of atopic dermatitis (AD) in a subject in need thereof, wherein the bacteria are not comprised in a faecal microbiota transplantation (FMT).

According to an aspect of some embodiments of the present invention there is provided a method of treating atopic dermatitis (AD) in a subject in need thereof comprising administering to the subject a therapeutically effective amount of between two and 100 species of bacteria, wherein at least two of the species are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594. SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, thereby treating the atopic dermatitis.

According to an aspect of some embodiments of the present invention there is provided a therapeutically effective amount of between two and 100 species of bacteria for use in the treatment of atopic dermatitis (AD) in a subject in need thereof, wherein at least two of the species are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis.

According to an aspect of some embodiments of the present invention there is provided a composition comprising a therapeutically effective amount of between two and 100 species of bacteria, wherein at least two of the species are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis.

According to some embodiments of the invention, the bacteria are of the genus Prevotella, Holdemanella and/or Megasphaera.

According to some embodiments of the invention, the bacteria are comprised in a well-defined preparation.

According to some embodiments of the invention, the bacteria comprising at least one of SGB_1636 Prevotella, SGB_1654 Prevotella, SGB_1644 Prevotella_copri, SGB_1640 Prevotella, SGB_1638 Prevotella, SGB_1626 Prevotella_copri;

SGB_1636 Prevotella, SGB_1654 Prevotella, SGB_1644 Prevotella_copri, SGB_1640 Prevotella, SGB_1638 Prevotella, SGB_1626 Prevotella_copri, SGB_1653 Prevotella; or

SGB_1636 Prevotella, SGB_1654 Prevotella, SGB_1644 Prevotella_copri, SGB_1640 Prevotella, SGB_1638 Prevotella, SGB_1626 Prevotella_copri, SGB_1624 Prevotella.

According to some embodiments of the invention, less than 10% of the bacteria in the composition are of the genus Clostridium.

According to some embodiments of the invention, less than 10% e of the bacteria in the composition are of the species Clostridium absonum, Clostridium argentinense, Clostridium baratii, Clostridium bifermentans, Clostridium botulinum, Clostridium butyricum, Clostridium cadaveris, Clostridium camis, Clostridium celatum, Clostridium chauvoei, Clostridium clostridioforme, Clostridium cochlearium, Clostridium difficile, Clostridium fallax, Clostridium felsineum, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium ramosum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and/or Clostridium villosum.

According to some embodiments of the invention, the administering comprises oral administration.

According to some embodiments of the invention, the administering comprises topical administration.

According to some embodiments of the invention, the bacteria are formulated for topical administration.

According to some embodiments of the invention, the bacteria are lyophilized or spray-dried.

According to some embodiments of the invention, the subject is an adult subject.

According to some embodiments of the invention, the subject is 3-18 years of age.

According to some embodiments of the invention, the subject exhibits moderate-to-severe AD with SCORAD score of >25.

According to some embodiments of the invention, the therapeutically effective amount of the bacteria causes at least 40% reduction in SCORAD score as compared to the SCOARD score of the subject prior to treatment with the bacteria.

According to some embodiments of the invention, the therapeutically effective amount of the bacteria causes a reduction in SCORAD score as compared to the SCOARD score of the subject prior to treatment with the bacteria, the reduction being maintained for at least 10 weeks following first cycle treatment with the bacteria.

According to some embodiments of the invention, the therapeutically effective amount of the bacteria results in colonization of the bacteria in the intestines of the subject.

According to some embodiments of the invention, the method further comprises treating the subject with an anti AD therapy other than the bacteria.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a scheme illustrating a study design according to some embodiments of the invention;

FIGS. 2A-C FIG. 2A. strain level dissimilarities by species. Box plots represent dissimilarities between different individuals from two healthy reference populations. Box limits represent the 25 and 75 quantiles and the whiskers represent the 5 and 95 quantiles. Dots are dissimilarities between post-FMT patient samples and their respective donor samples, colored by patient. Dissimilarities are clipped from bottom to the detection threshold (5e-5, dashed line). FIG. 2B. Number of detected transmission events by species, colored by patient. FIG. 2C. Species-Donor transmission table. The top row summarizes the number of transmitted species for every patient with donor indication above—“main donor (secondary donor)”.

FIG. 3 is a graph showing changes in patients' SCORAD across study time.; Black line represents the average SCORAD of allegeable patients (with patient 6 excluded). Vertical lines indicate placebo treatments and FMTs.

FIG. 4 is a graph showing SCORAD changes in long-term follow-up patients 2 and 11 SCORAD across time. Vertical lines indicate placebo treatments and FMTs.

FIGS. 5A-B show changes in gut bacterial strains of patients following FMT. FIG. 5A. Dissimilarity across time following FMT in bacterial strains of samples from patient 11 compared to baseline. Grey lines represent different species (left axis), black line represents the average dissimilarity (right axis). Vertical lines indicate placebo treatments and FMTs. Dissimilarities are clipped from bottom to the detection threshold (5e-5, horizontal grey dashed line). FIG. 5B. Same as (FIG. 5A) but when comparing bacterial strains of samples from patient 11 to the sample of donor 1 whose stool was used in the FMT of patient 11.

FIG. 6 shows that patients' samples become similar to donor samples following FMT. t-SNE (t-distributed Stochastic Neighbor Embedding) analysis based on sample-dissimilarity matrix of patients with available pre-FMT and post-FMT samples (N=8) and donors' samples (N=3), showing shift of patient samples towards their donor's cluster. Arrows demonstrate the path from baseline samples (square markers), through placebo samples (diamond markers) to post-FMT samples (circle markers). Color scale matches the patient's main donor. Empty ellipses encompass the donors' samples and filled ellipses encompass patient post-FMT samples.

FIG. 7 is a graph showing Correlation between clinical improvement and similarity in bacterial strains of patients and donors following FMT. Dots correspond to individual patient samples, plotted by their dissimilarity to their donor (x-axis) versus the reduction of the SCORAD score from baseline at the time of the sample (y-axis). Arrows demonstrate the path from placebo samples (diamond shape markers) to post-FMT samples. Ellipses encompass.

FIG. 8 is a table showing donor stool samples that were used for FMT capsules and stool samples that were collected from the patients during the study period that were sequenced into metagenomics reads using Illumina NextSeq.

### DESCRIPTION OF SPECIFIC EMBODIMENTS OF THE INVENTION

The present invention, in some embodiments thereof, relates to treatment of atopic dermatitis.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Atopic dermatitis (AD) is a remitting relapsing chronic eczematous pruritic skin disease affecting more than 20% of children and up to 10% of adults. It is a multifactorial disease influenced by genetic, immunologic, epidermal dysfunction, and environmental factors.

Whilst conceiving embodiments of the present invention, the present inventors sought to identify gut microbiome-derived bacterial species that can be used for the treatment of AD.

The present inventors performed a prospective, single-blinded, placebo-controlled cross-over pilot study among ten adult patients who had moderate-to-severe atopic dermatitis, insufficiently responsive to topical and systemic treatment. All patients received 2 placebo fecal transplantations followed by 4 FMTs from healthy donors each 2 weeks apart. The severity of AD and the fecal microbiome profile were evaluated by the Scoring Atopic Dermatitis Score (SCORAD score) at the beginning of the study, before every FMT, and 1-8 months after the last FMT. Results of this study show that there was no significant change in the severity of AD following the two placebo transplants (SCORAD augmentation 2.5%±22.1%, Wilcoxon P>0.85). Two weeks following all four doses of FMT there was a significant reduction in the SCORAD score (59.2%±34.9%, Wilcoxon P<0.02). The maximal score reduction during the intervention period and the follow-up period was even higher (84%±7%, Wilcoxon P<0.008). Metagenomic analysis of the fecal microbiota of patients and donors showed transmission of bacterial strains from donors to patients, and some donor strains were transmitted to multiple patients with the Prevotella genus standing out. No adverse events were recorded during the study and follow-up period.

It is therefore suggested that FMT is safe and effective as a therapeutic intervention for AD, such as those subjects suffering from a moderate-to-severe disease.

Thus, according to an aspect of the invention there is provided a method of treating atopic dermatitis (AD) in a subject in need thereof comprising administering to the subject a therapeutically effective amount of at least two species of bacteria, wherein said at least two species of said bacteria are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, thereby treating the atopic dermatitis, wherein said bacteria are not comprised in a faecal microbiota transplantation (FMT).

According to an aspect of the invention there is provided a therapeutically effective amount of at least two species of bacteria, wherein said at least two species of said bacteria are selected from the group consisting SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, for use in the treatment of atopic dermatitis (AD) in a subject in need thereof, wherein said bacteria are not comprised in a faecal microbiota transplantation (FMT).

According to an aspect of the invention there is provided a method of treating atopic dermatitis (AD) in a subject in need thereof, comprising administering to the subject a therapeutically effective amount of between two and 100 species of bacteria, wherein at least two of the species are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, thereby treating the atopic dermatitis.

According to an additional or alternative embodiment, there is provided a therapeutically effective amount of between two and 100 species of bacteria, for use in the treatment of atopic dermatitis (AD) in a subject in need thereof, wherein at least two of said species are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis.

According to an additional or alternative embodiment, there is provided a composition comprising a therapeutically effective amount of between two and 100 species of bacteria, wherein at least two of the species are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB 5045 Eubacterium ventriosum and SGB 9283 Sutterella wadsworthensis.

According to an embodiment of the invention, the bacteria are of the genus Prevotella, Holdemanella and/or Megasphaera.

According to an embodiment of the invention, the bacteria are comprised in a well-defined preparation.

According to an embodiment of the invention, the bacteria comprises at least one of SGB_1636 Prevotella, SGB_1654 Prevotella, SGB_1644 Prevotella_copri, SGB_1640 Prevotella, SGB_1638 Prevotella, SGB_1626 Prevotella_copri;

SGB_1636 Prevotella, SGB_1654 Prevotella, SGB_1644 Prevotella_copri, SGB_1640 Prevotella, SGB_1638 Prevotella, SGB_1626 Prevotella_copri, SGB_1653 Prevotella; or

SGB_1636 Prevotella, SGB_1654 Prevotella, SGB_1644 Prevotella_copri, SGB_1640 Prevotella, SGB_1638 Prevotella, SGB_1626 Prevotella_copri, SGB_1624 Prevotella.

According to an embodiment of the invention, at least 20%, 30% or 40% of the administered bacteria are of the genus Prevotella, also referred to as a Prevotella enriched composition.

The therapeutic improvement may be associated with direct or indirect fecal material derived bacteria.

Bacteria that are “directly isolated from a fecal material” does not result from any culturing or other process that results in or is intended to result in replication of the population after obtaining the fecal material (also termed herein as “FMT”).

According to a specific embodiment, the bacteria are comprised in a well-defined preparation.

As used herein “well-defined” means a bacterial culture in which the identity of the microbes is known a priori. Although there may be some level of contamination, this typically does not exceed 10% of the microbial population and the rest of the at least 90% of the microbes in the well-defined preparation are known prior to administration.

As used herein the term “atopic dermatitis” or “AD” refers to a chronic relapsing and remitting inflammatory skin disorder characterized by intense pruritus (e.g., severe itch), xerosis (e.g., abnormally dry skin), erythematous crusting rash, lichenification, an impaired skin barrier and by scaly and dry eczematous lesions.

Atopic dermatitis includes, but is not limited to, AD caused by or associated with epidermal barrier dysfunction, allergy (e.g., allergy to certain foods, pollen, mold, dust mite, animals, etc.), radiation exposure, and/or asthma. In many cases, chronic AD lesions include thickened plaques of skin, lichenification and fibrous papules.

As used herein the term “subject” refers to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject is a human. In some embodiments, an “individual” or “patient” or “subject” herein is any single human subject eligible for treatment who is experiencing or has experienced atopic dermatitis.

According to a specific embodiment, the human subject is an adult, i.e., 18 years of age or above, e.g., greater than 20, 25, 30, 35, 40, 45, 50, 55, or 60 years of age. The subject can be a senior, such as a subject who is greater than 65, 70, 75, or 80 years of age.

According to another specific embodiment, the human subject is pediatric, i.e., below 18 years of age (e.g., 2-12, 12-18, 2-11, 12-18, 2-4, 2-6, 6-12, 12-18, 1-4, 5-8, 9-15 years old).

In some embodiments, the subject is a child, such as a subject that is 11, 10, 9, 8, 7, 6, 5, 4, 3, 2 or 1 year(s) of age or less. The subject can be an infant, such as a subject of less than 1 year of age.

The subject can be immunocompromised or can have an intact immune system (immunocompetent).

The subject may be treated with various conventional or non-conventional medications/treatments for AD prior to treatment according to some embodiments of the present invention, some are detailed hereinbelow.

Alternatively, the subject may be an untreated subject (e.g., at least for the last 6-12 months prior to the initiation of the treatment according to some embodiments of the invention), accordingly such a subject is also termed as “naive”.

Atopic dermatitis can be diagnosed using “Hanifin/Rajka criteria.” Hanifin/Rajka diagnostic criteria are described in Acta Derm Venereol (Stockh) 1980; Suppl 92:44-7. To establish a diagnosis of atopic dermatitis the patient requires the presence of at least 3 “basic features” and 3 or more minor features listed below. The basic features include pruritus, typical morphology and distribution such as flexural lichenification or linearity, chronic or chronically-relapsing dermatitis, and personal or family history of atopy, such as asthma, allergic rhinitis, atopic dermatitis. The minor features include xerosis, ichthyosis, palmar hyperlinearity, or keratosis pilaris, immediate (type 1) skin-test reactivity, elevated serum IgE, early age of onset, tendency toward cutaneous infections (especially Staph. aureus and Herpes simplex), impaired cell-mediated immunity, tendency toward non-specific hand or foot dermatitis, nipple eczema, cheilitis, recurrent conjunctivitis, Dennie-Morgan infraorbital fold, keratoconus, anterior subcapsular cataracts, orbital darkening, facial pallor/facial erythema, pityriasis alba, anterior neck folds, and itch when sweating. Additional minor criteria include intolerance to wool and lipid solvents, perifollicular accentuation, food intolerance, course influenced by environmental or emotional factors, and white dermographism/delayed blanch.

The severity of atopic dermatitis can be determined by “Rajka/Langeland criteria,” as described in Rajka G and Langeland T, Acta Derm Venereol (Stockh) 1989; 144(Suppl):13-4. Three disease severity assessment categories are scored 1 to 3: I) extent of the body area involved, II) course, e.g., more or less than 3 months during one year or continuous course, and III) intensity, ranging from mild itch to severe itch, usually disturbing night's sleep. Scores of 1.5 or 2.5 are allowed. Overall disease severity is determined by the sum of individual scores from the three disease assessment categories and the severity is determined by the sum of these scores with mild defined as a total score of 3-4, moderate as score of 4.5-7.5, and severe as a total score of 8-9.

The term “Atopic Dermatitis Disease Severity Outcome Measure” or “ADDSOM” means a determination of certain signs, symptoms, features or parameters that have been associated with atopic dermatitis and that can be quantitatively or qualitatively assessed. Exemplary ADDSOMs include, but are not limited to, “Eczema Area and Severity Index” (EAST), “Severity Scoring of Atopic Dermatitis” (SCORAD), “Investigator Global Assessment” (IGA), “Investigator Global Assessment of Signs” (IGSA), Rajka/Langeland Atopic Dermatitis Severity Score, and Patient-Reported Outcomes including, but not limited to, Pruritus Visual Analog Scale (an aspect of disease severity assessed as part of SCORAD), Sleep Loss Visual Analog Scale (an aspect of disease severity assessed as part of SCORAD), Atopic Dermatitis Symptom Diary (ADSD), Atopic Dermatitis Impact Questionnaire (ADIQ), Dermatology Life Quality Index (DLQI) (Finlay and Khan, Clin Exper Dermatol 1994; 19:210), and 5-D Itch Scale (Elman et al., Br J Dermatol 2010; 162(3):587-593).

The “Eczema Area and Severity Index” or “EAST” is a validated measure used in clinical settings to assess the severity and extent of AD, Hanifin et al., Exp Dermatol 2001; 10:11-18. Four individual body regions are assessed by a clinician or other medical professional: head and neck (H&N), upper limbs (UL; includes the external axillae and hands), trunk (includes the internal axillae and groin), and lower limbs (LL; includes the buttocks and feet). For each body region, the affected body surface area (BSA) is assessed and assigned a score of 0 to 6 (or optionally 0-7 where 0 equals no eruption) for the percentage of affected BSA (0%-100%); each region is individually rated for the average degree of severity (0-3: none, mild, moderate, severe), with half steps allowed, for each of four clinical signs: erythema, induration-papulation, excoriations, and lichenification. A summed score of 0 to 12 is assigned to each body region; a total body region score is assigned based on the sum of the individual clinical signs score (maximum=12).times.affected area score (maximum=6).times.the body-region index (H&N—0.1, UL—0.2, trunk—0.3, LL—0.4). A total score (0-72) is assigned based on the sum of total scores for each of the four body region scores.

The “Investigator Global Assessment” or “IGA” is an assessment measure used in clinical settings to determine the severity of AD and clinical response to treatment based on a five-point scale. A score of 0 (clear) means there are no inflammatory signs of atopic dermatitis and a score of 1 (almost clear) means there is just perceptible erythema and just perceptible papulation induration. Scores of 2, 3, or 4 (mild, moderate, severe) are based on the severity of erythema, papulation induration, oozing and crusting. The “Investigator Global Assessment of Signs” or “IGSA” uses a lesional assessment grade ranging from clear to severe based on an evaluation of erythema, edema, lichenified plaques or papules, and excoriations. In addition, the assessed lesional grade may be upgraded or downgraded based on the extent and location of the skin lesion.

The “Severity Scoring of Atopic Dermatitis” or “SCORAD” is a clinical assessment of the severity of AD developed by the European Task Force on Atopic Dermatitis (consensus report, Dermatology 1993; 186:23-31). Three aspects of disease severity are scored: (i) the extent of body area affected by inflammation with score assessed between 0-100, assigned as “A” in the overall total score, (ii) the intensity of six clinical signs, erythema, edema/population, oozing/crusting, excoriation, lichenification, and dryness, each assigned a score of 0-3 based on severity (absent, mild, moderate, severe) for a total score ranging from 0-18, assigned as “B” in the overall total score, and (iii) two subjective measures that use patient-reported outcomes, the pruritus visual analog score (ranging from 0 [no itch] to 10 [the worst imaginable itch]) and the sleep loss visual analog score (ranging from 0 [no sleep loss] to 10 [the worst imaginable sleep loss]), each the average of the last three days or nights, assigned as “C” in the overall total score. The overall total score (0-103) is determined according to the formula: A/5+(7B/2)+C.

According to SCORAD mild is typically defined as below 25, moderate is typically defined as 25-50 and severe is typically defined as above 50.

A “species” can be defined by a combination of core genome phylogenetic analysis and ANI which provide an appropriate method for bacterial species delineation, whereby bacterial species are defined as monophyletic groups of isolates with genomes that exhibit at least 95% pair-wise ANI.

When referring to the number of bacterial species it is meant to different bacterial species.

The composition may include other microbes e.g., yeast, fungi and more.

The compositions of this aspect of the present invention may contain 2, 3, 4, 5, 6, 7, 8, 9, 10, 11, 12, 13, 14, 15, 16, 17, 18, 19 or 20 different species of bacteria.

According to a specific embodiment, the methods and/or compositions make use of 2-100, 2-50, 2-40, 2-30 different bacterial species. e.g., 2-50, 2-40, 2-30, 2-20, 2-18, 2-15, 2-12, 2-10, 2-8, 2-5, 2-4, 2-3, 3-20, 3-18, 3-15, 3-12, 3-10, 3-8, 3-5, 3-4, 4-20, 4-18, 4-15, 4-12, 4-10, 4-8, 4-5, 5-20, 5-18, 5-15, 5-12, 5-10, 5-8, 6-20, 6-18, 6-15, 6-12, 6-10, 6-8, 7-20, 7-18, 7-15, 7-12, 7-10, 7-8, 8-20, 8-18, 8-15, 8-12, 8-10, 9-20, 9-18, 9-15, 9-12, 9-10, 10-20, 10-18, 10-15, 10-12, 11-12, 12-15, 12-18, 12-20, 14-16, 14-18, 14-20 or 15-20, 10-50, 12-50, 14-50, 16-50, 18-50, 20-50, 25-50, 30-50, 35-50 or 40-50, 10-40, 12-40, 14-40, 16-40, 18-40, 20-40, 25-40, 30-40, 35-4-10, 10-30, 12-30, 14-30, 16-30, 18-30, 20-30, 25-30.

In one embodiment, the bacteria are provided in a composition (e.g., one composition, where all the strains are combined in a single composition; or more, where at least some strains are provided in different compositions), whereby according to some embodiments, the composition is a probiotic composition.

As used herein, the phrase “probiotic composition” refers to a composition which comprises live micro-organisms, which when administered in adequate amounts, confer a health benefit on the host. Probiotics are typically alive when administered, have viability and reproducibility based on in vivo testing, and during use and storage.

In one embodiment, at least 20° %, at least 30%, at least 40%, at least 50%, at least 60%. at least 70%, at least 80%, at least 90% of the bacteria of the composition are viable.

According to some embodiments of the invention, the present invention envisages treatment with-, or compositions that comprise at least two of the following species: SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis.

According to some embodiments of the invention, the present invention envisages treatment with-, or compositions that comprise at least three of the following species: SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis.

According to some embodiments of the invention, the present invention envisages treatment with-, or compositions that comprise at least four of the following species: SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38. SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis.

According to some embodiments of the invention, the present invention envisages treatment with-, or compositions that comprise at least five of the following species: SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis.

According to some embodiments of the invention, the present invention envisages treatment with-, or compositions that comprise all of the following species: SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB 5045 Eubacterium ventriosum and SGB 9283 Sutterella wadsworthensis.

In one embodiment, the composition comprises no more than three, no more than four, no more than five, no more than six, no more than seven, no more than eight, no more than nine, no more than ten bacterial species.

In one embodiment, at least 50%, 55%, 60%, 65%, 70%. 75%, 80%, 85%, 90%, 95%, or even 99% of the bacteria of the contemplated compositions are of the genus Prevotella.

In one embodiment, at least 30%, 35%, 40%, 45%, 50%, 55%, 60%. 65% or even 70% of the bacteria in the composition are of the species. at least one of SGB_1636 Prevotella, SGB_1654 Prevotella, SGB_1644 Prevotella_copri, SGB_1640 Prevotella, SGB_1638 Prevotella, SGB_1626 Prevotella_copri; SGB_1636 Prevotella, SGB_1654 Prevotella, SGB_1644 Prevotella_copri, SGB_1640 Prevotella, SGB_1638 Prevotella, SGB_1626 Prevotella_copri, SGB_1653 Prevotella; or SGB_1636 Prevotella, SGB_1654 Prevotella, SGB_1644 Prevotella_copri, SGB_1640 Prevotella, SGB_1638 Prevotella, SGB_1626 Prevotella_copri, SGB_1624 Prevotella.

In one embodiment, at least 0.5%, 1%, 2%, 3%, 4%, 5%, 6%, 7%, 8%, 9%, 10% of the bacteria in the composition are of the species:

at least one of SGB_1636 Prevotella, SGB_1654 Prevotella, SGB_1644 Prevotella_copri, SGB_1640 Prevotella, SGB_1638 Prevotella, SGB_1626 Prevotella_copri;

SGB_1636 Prevotella, SGB_1654 Prevotella, SGB_1644 Prevotella_copri, SGB_1640 Prevotella, SGB_1638 Prevotella, SGB_1626 Prevotella_copri, SGB_1653 Prevotella; or

SGB_1636 Prevotella, SGB_1654 Prevotella, SGB_1644 Prevotella_copri, SGB_1640 Prevotella, SGB_1638 Prevotella, SGB_1626 Prevotella_copri, SGB_1624 Prevotella.

According to a specific embodiment, the treatments described herein and/or the compositions comprise less than 20%, 10%, 8%, 5 or even less than 1% bacteria of the genus Clostridium.

According to a specific embodiment, the treatments described herein and/or the compositions comprise less than 20%, 10%, 8%, 5% or even less than 1% the bacterial species Clostridium absonum, Clostridium argentinense, Clostridium baratii, Clostridium bifermentans, Clostridium botulinum, Clostridium butyricum, Clostridium cadaveris, Clostridium camis, Clostridium celatum, Clostridium chauvoei, Clostridium clostridioforme, Clostridium cochlearium, Clostridium difficile, Clostridium fallax, Clostridium felsineum, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium ramosum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and/or Clostridium villosum.

The compositions of the present invention may be pH adjusted. They may be packaged into single dosage units for ease of administration or multiple dosage units for each bacterial species.

In some embodiments, the bacteria or compositions comprising same can be lyophilized or spray dried and stored frozen or in a sterile container, for resuspension immediately prior to use. The bacterial compositions can be resuspended with sterile water, a weak acidic solution, gel, or buffer.

The subject may be treated with an antibiotic prior to treatment with the bacterial composition. The antibiotic may be provided for at least 2 days, at least 3 days, at least 4 days, at least 5 days, at least 6 days, at least seven days or longer.

In yet another embodiment, the bacteria described herein are incorporated into a pharmaceutical product or composition. Pharmaceutical compositions comprise a therapeutically effective amount of the bacteria (e.g., composition) described herein and typically one or more pharmaceutically acceptable carriers or excipients (which are discussed below).

In some embodiments the pharmaceutical composition is for oral, topical, nasal, respiratory, parenteral, intraperitoneal, intravenous, intraarterial, transdermal, sublingual, intramuscular, rectal, transbuccal, intraocular, subcutaneous, intraadiposal, intraarticular or intrathecal administration. The disclosure contemplates formulations of the bacteria described herein that are, in some embodiments, powdered, tableted, encapsulated (e.g., in one or more e.g., two or more layers) or otherwise formulated for oral administration.

The bacteria of the present invention may also be formulated in a rectal composition such as a suppository or a retention enema containing conventional suppository bases such as cocoa butter or other glycerides.

The compositions may be provided as pharmaceutical compositions, nutraceutical compositions (e.g., a dietary supplement), or as a food or beverage additive, as defined by the U.S. Food and Drug Administration (FDA). The dosage form for the above compositions are not particularly restricted. For example, liquid solutions, suspensions, emulsions, tablets, pills, capsules, sustained release formulations, powders, suppositories, liposomes, microparticles, microcapsules, sterile isotonic aqueous buffer solutions, enemas, suppositories and the like are all contemplated as suitable dosage forms.

The compositions typically include one or more suitable diluents, fillers, salts, disintegrants, binders, lubricants, glidants, wetting agents, controlled release matrices, colorings, flavoring, carriers, excipients, buffers, stabilizers, solubilizers, commercial adjuvants, and/or other additives known in the art.

Any pharmaceutically acceptable (i.e., sterile and acceptably non-toxic as known in the art) liquid, semisolid, or solid diluent that serves as a pharmaceutical vehicle, excipient, or medium can be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, .alpha.-lactose, anhydrous lactose, cellulose, sucrose, dextrose, sorbitol, modified dextrans, gum acacia, and starch.

Pharmaceutically acceptable fillers can include, for example, lactose, microcrystalline cellulose, dicalcium phosphate, tricalcium phosphate, calcium sulfate, dextrose, mannitol, and/or sucrose. Salts, including calcium triphosphate, magnesium carbonate, and sodium chloride, may also be used as fillers in the pharmaceutical compositions.

Binders may be used to hold the composition together to form a hard tablet. Exemplary binders include materials from organic products such as acacia, tragacanth, starch and gelatin. Other suitable binders include methyl cellulose (MC), ethyl cellulose (EC) and carboxymethyl cellulose (CMC).

In some embodiments, an enriched food product further comprises a bioavailability enhancer, which acts to increase the absorption of the sorbable natural product(s) by the body. Bioavailability enhancers can be natural or synthetic compounds. In one embodiment, the enriched food product comprising the composition described herein further comprises one or more bioavailability enhancers in order to enhance the bioavailability of the bioactive natural product(s).

Natural bioavailability enhancers include ginger, caraway extracts, pepper extracts and chitosan. The active compounds in ginger include 6-gingerol and 6-shogoal. Caraway oil can also be used as a bioavailability enhancer (U.S. Patent Application 2003/022838). Piperine is a compound derived from pepper (Piper nigrum or Piper longum) that acts as a bioavailability enhancer (see U.S. Pat. No. 5,744,161). Piperine is available commercially under the brand name Bioperine® (Sabinsa Corp., Piscataway, N.J.). In some embodiments, the natural bioavailability enhancers is present in an amount of from about 0.02% to about 0.6% by weight based on the total weight of enriched food product.

Examples of suitable synthetic bioavailability enhancers include, but are not limited to surfactants including those composed of PEG-esters such as are commercially available under the tradenames: Gelucire®, Labrafil®, Labrasol®, Lauroglycol®, Pleurol Oleique® (Gattefosse Corp., Paramus, N.J.) and Capmul® (Abitec Corp., Columbus, Ohio).

The amount and administration regimen of the composition is based on various factors relevant to the purpose of administration and severity of symptoms, for example human or animal age, sex, body weight, hormone levels, or other nutritional need of the human or animal. Repeated administration is also contemplated in order to prevent exacerbation of symptoms and optionally keep the subject at a stage of remission.

A typical regimen may comprise multiple doses of the composition. In one embodiment, the composition is administered once per day, thrice a week, twice a week, once a week, once per two weeks, once per month, once per two months. The composition may be administered to an individual at any time. In some embodiments, the composition is administered concurrently, or prior to or at the consumption of a meal.

According to a specific embodiment, the bacteria are provided by topical administration. These compositions include a pharmaceutically acceptable carrier, and optionally include additional compounds. In some embodiments, the pharmaceutical composition includes additional active and/or inactive materials in order to produce a final product, which may be in single dosage unit or in a multi-dose format.

In some embodiments the pharmaceutical composition can include one or more of a buffering agent, a preservative, a stabilizer, a binder, a compaction agent, a lubricant, a dispersion enhancer and/or a coloring agent. Non-limiting examples of suitable buffering agents include sodium citrate, magnesium carbonate, magnesium bicarbonate, calcium carbonate, and calcium bicarbonate. Non-limiting examples of suitable preservatives include antioxidants, such as alpha-tocopherol and ascorbate, parabens, chlorobutanol, and phenol. Non-limiting examples of suitable binders include sucrose, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. A pH buffering agent(s) can, if employed and when dissolved in an aqueous component of the composition, provide a pH in the range of 5 to 7 (e.g. about pH 5.5).

The pharmaceutical composition can include other ingredients, such as to sustain growth of the bacteria. In some embodiments, the pharmaceutical composition can include a nutrient. In some embodiments the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. A carbohydrate can be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates can contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose (e.g., 2′-fluororibose, deoxyribose, and hexose). Carbohydrates can exist in many different forms, for example, conformers, cyclic forms, acyclic forms, stereoisomers, tautomers, anomers, and isomers.

In some embodiments the composition includes at least one lipid. A “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In additional embodiments the composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

Various other additives may be included in the compositions. These include, but are not limited to, antioxidants, astringents, perfumes, preservatives, emollients, pigments, dyes, humectants, propellants, and sunscreen agents, as well as other classes of materials whose presence may be pharmaceutically or otherwise desirable. Non-limiting examples of optional additives are as follows: preservatives such as sorbate; solvents such as isopropanol and propylene glycol; astringents such as menthol and ethanol; emollients such as polyalkylene methyl glucosides; humectants such as glycerine; emulsifiers such as glycerol stearate, PEG-100 stearate, polyglyceryl-3 hydroxylauryl ether, and polysorbate 60; sorbitol and other polyhydroxyalcohols such as polyethylene glycol; sunscreen agents such as octyl methoxyl cinnamate (available commercially as Parsol MCX) and butyl methoxy benzoylmethane (available under the tradename Parsol 1789); antioxidants such as ascorbic acid (vitamin C), a-tocopherol (Vitamin E), .beta.-tocopherol, .gamma.-tocopherol, .delta.-tocopherol, .epsilon.-tocopherol, .zeta.-tocopherol, Z^-tocopherol, mtocopherol, and retinol (vitamin A); essential oils, ceramides, essential fatty acids, mineral oils, vegetable oils (e.g., soya bean oil, palm oil, liquid fraction of rhea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes.

Other additives include materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-tchloro-2-hydroxy diphenyl ether (triclosan) and benzoic acid. Further additives include anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof.

In other embodiments, the composition can include alpha hydroxy acids, alpha ketoacids, polymeric hydroxyacids, moisturizers, collagen, marine extract, and antioxidants such as ascorbic acid (vitamin C) and/or a-tocopherol (Vitamin E). Sunscreens may be included. Additional, components such as enzymes, herbs, plant extracts, glandular or animal extracts can be added to the composition. The amounts of these various additives are those conventionally used in the cosmetics field, and range, for example, from about 0.01% to about 20% of the total weight of the topical formulation.

The compositions can also include antimicrobial agents, to prevent spoilage upon storage, i.e., to inhibit growth of microbes such as yeasts and molds. Measures are taken not to use antibiotics that affect the therapeutic bacteria.

The compositions can also contain irritation-mitigating additives to minimize or eliminate the possibility of skin irritation or skin damage resulting from the chemical entity to be administered, or other components of the composition. Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphiphilic amines; ammonium chloride; N-acetylcysteine, capsaicin, and chloroquine.

Further suitable pharmacologically active agents that may be incorporated into the present formulations in certain embodiments and thus topically applied along with the active agent include, but are not limited to, the following: agents that improve or eradicate pigmented or non-pigmented age spots, keratoses, and wrinkles; local anesthetics and analgesics; corticosteroids; retinoids; and hormones. Some examples of topical pharmacologically active agents include acyclovir, amphotericins, chlorhexidine, clotrimazole, ketoconazole, econazole, miconazole, metronidazole, minocycline, phenytoin, para-amino benzoic acid esters, octyl methoxycinnamate, octyl salicylate, oxybenzone, dioxybenzone, tocopherol, tocopheryl acetate, zinc pyrithione, diphenhydramine, pramoxine, lidocaine, procaine, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clohetasol propionate, minoxidil, dipyridamole, diphenylhydantoin, benzoyl peroxide, 5-fluorouracil, tacrolimus, and topical steroids such as alclometasone, amcinonide, betamethasone, clobetasol, desonide, desoximetasone, diflorasone, flucinonide, flurandrenolide, halobetasol, halcinonide, hydrocortisone, and/or triamcinolone.

Although topical formulations, such as creams and salves formulated for dermal delivery are contemplated, the delivery systems can include time-release, delayed release or sustained release delivery systems. Such systems can avoid repeated administrations of the compositions, increasing convenience to the subject and the physician. Many types of release delivery systems are available and known to those of ordinary skill in the art. Specific examples include, but are not limited to: (a) erosional systems such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480.

The delivery system can include collagen, fibrin, or a membrane extract, such as a basal membrane extract, for example wherein the composition is formulated for administration to the skin. Suitable basement membrane extracts include a biologically active polymerizable extract containing in parts by weight about 60-85% laminin, 5-30% collagen IV, 1-10% nidogen, 1-10% heparan sulfate proteoglycan and 1-5% entactin (see U.S. Pat. No. 4,829,000, incorporated herein by reference, which discloses BME compositions as well as methods for producing these compositions). BME can support normal growth and differentiation of various cell types including epithelial cells when cultured. Basal membrane extracts are well known in the art and are commercially available.

For treatment of the skin, a therapeutically effective amount of the composition can be locally administered to the affected area. The pharmacological compositions disclosed herein facilitate the use of the bacteria for the treatment of atopic dermatitis. Such a composition can be suitable for delivery of the active ingredient to any suitable subject, such as but not limited to, a human subject, and can be manufactured in a manner that is itself known, e.g., by means of conventional mixing, dissolving, granulating, emulsifying, encapsulating, entrapping or lyophilizing processes. Pharmacological compositions can be formulated in a conventional manner using one or more pharmacologically (e.g., physiologically or pharmaceutically) acceptable carriers, as well as optional auxiliaries that facilitate processing of the active compounds into preparations which can be used pharmaceutically, as discussed above.

The composition can be applied to the skin, such as at lesion areas and round lesion area, or at areas of intact skin (non-lesion areas) to prevent lesions for forming. The composition can be used to reduce lesion size. The composition can be applied daily. The composition can be applied 1, 2, 3, 4, or 5 time per day. The composition can be applied every other day, or 1, 2, 3, 4, 5, 6, or 7 times per week. The composition can be applied weekly.

Methods of producing topical pharmaceutical compositions such as creams, ointments, lotions, sprays and sterile aqueous solutions or suspensions are well known in the art. Suitable methods of preparing topical pharmaceutical compositions are described, for example in PCT Publication No, WO 95/10999, PCT Publication No, WO2012150269, U.S. Pat. No. 6,974,585, and PCT Publication No. WO 2006/048747, all incorporated herein by reference. The composition can include an aqueous carrier, and be applied as a spray to the skin.

Optionally, a composition can include a pharmaceutically acceptable viscosity enhancer and/or film former. A viscosity enhancer increases the viscosity of the formulation so as to inhibit its spread beyond the site of application. Balsam Fir (Oregon) is an example of a pharmaceutically acceptable viscosity enhancer of use with the bacteria.

A film former, when it dries, forms a protective film over the site of application. The film inhibits removal of the active ingredient and keeps it in contact with the site being treated. An example of a film former that is suitable for use in this invention is Flexible Collodion, USP. As described in Remington: The Science and Practice of Pharmacy, 19th Ed. (Easton, Pa.: Mack Publishing Co., 199.5), at page 1530, collodions are ethyl ether/ethanol solutions containing pyroxylin (a nitrocellulose) that evaporate to leave a film of pyroxylin. A film former may act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Lotions, are preparations to be applied to the skin surface without friction, and are typically liquid or semiliquid preparations in which particles, including the active agent, are present in a water or alcohol base. Lotions are usually suspensions of solids, and preferably, comprise a liquid oily emulsion of the oil-in-water type. Lotions can be used for treating large body areas, because of the ease of applying a more fluid composition. It is generally necessary that the insoluble matter in a lotion be finely divided.

Lotions typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solutes) in a liquid such that the molecules of the dissolved substance are dispersed among those of the solvent. The solution may contain other pharmaceutically or cosmetically acceptable chemicals to buffer, stabilize or preserve the solute. Common examples of solvents used in preparing topical solutions are ethanol, water, propylene glycol or any other acceptable vehicles. These can be applied in any manner, such as spraying them on the skin, painting them on the skin, or wetting a bandage with the solution.

Gels are semisolid, suspension-type systems. Single-phase gels contain organic macromolecules distributed substantially uniformly throughout the carrier liquid, which is typically aqueous, but also, preferably, contain an alcohol, and, optionally, an oil. Some “organic macromolecules,” of use, specifically gelling agents, are crosslinked acrylic acid polymers such as the “carbomer” family of polymers, e.g., carboxypolyalkylenes that re commercially available as CARBOPOL®. Also of use are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof. These gels are of use in the methods disclosed herein.

Ointments can also be used in the disclosed methods. Ointments are semisolid preparations that are typically based on petrolatum or other petroleum derivatives. The specific ointment base to be used, as will be appreciated by those skilled in the art, is one that will provide for a number of desirable characteristics, e.g., emolliency or the like. An ointment base is generally inert, stable, nonirritating, and nonsensitizing. Ointment bases are grouped in four classes: oleaginous bases; emulsifiable bases; emulsion bases; and water-soluble bases (see Remington: The Science and Practice of Pharmacy, 19th Ed. (Easton, Pa.: Mack Publishing Co., at pages 1399-1404), Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, acetyl alcohol, glyceryl monostearate, lanolin, and stearic acid. Water-soluble ointment bases are prepared from polyethylene glycols of varying molecular weight.

Pastes are semisolid dosage forms in which the active agent is suspended in a suitable base, and are also of use. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

A topical composition can any form suitable for application to the body surface, such as a cream, lotion, sprays, solution, gel, ointment, paste, plaster, paint, bioadhesive, bandage, sprays, suspensions or the like, and/or may be prepared so as to contain liposomes, micelles, and/or microspheres. A topical composition can be used in combination with an occlusive overlayer so that moisture evaporating from the body surface is maintained within the formulation upon application to the body surface and thereafter.

A cream, lotion, gel, ointment, paste or the like may be spread on the affected surface. A solution may be applied in the same way, but more typically will be applied with a dropper, swab, sprayer or the like, and carefully applied to the affected areas. The composition can be applied directly to the target location, for example in a topical preparation such as an ointment, or as a part of a dressing or a bandage. The composition can be formulated as a unit dosage, for administration by any device for administration to the skin. The unit dosage may be a reservoir of the active agent in a carrier, for example an adhesive carrier capable of adhering to the skin for a desired period of time such as at least a day or more.

The pharmaceutical compositions are of use for the treatment of atopic dermatitis. Thus, in some embodiments, application results in reduced lesion size, reduce number of lesions, and/or a reduction in symptoms. The application of these pharmaceutical compositions can reduce S. aureus in the skin of the subject being treated. The application of the pharmaceutical composition can provide enhanced barrier function of the skin as measured by trans-epidermal water loss.

Atopic dermatitis occurs as flare-ups, and there can be periods of remission. The topical application can reduce reoccurrences, so that additional incidents of atopic dermatitis are reduced in number, intensity, or frequency. The topical application can increase the time of remission, such as the length of time between incidents. In some embodiments, an additional incident of atopic dermatitis will not occur for 1, 2, 3, 4, 5, 6, 7, 8, 9, 10, 11, or 12 weeks following application. In an additional embodiment, an additional incident of atopic dermatitis will not occur for 1, 2, 3, 4, 5, 6, 7, 8, 9, 10, 11, or 12 months following the topical application.

Treatment with the bacteria of some embodiments of the invention can be combined with an anti AD therapy other than the bacteria.

Examples include but are not limited to immunotherapeutics, anti-angiogenic agents, cytokines, hormones, antibodies, polynucleotides, photodynamic therapeutic agents, non-steroid anti-inflammatory medications, anti-histamines, alpha-adrenergic agonists, steroids (e.g., corticosteroids), and any combination thereof.

The term “corticosteroid” includes, but is not limited to, topical corticosteroids. Exemplary topical corticosteroids include triamcinolone acetonide, typically formulated at a concentration of 0.1% in a cream, and hydrocortisone, typically formulated at a concentration of 1% or 2.5% in a cream. Certain topical corticosteroids are considered very high potency such as, for example, betamethasone dipropionate, clobetasol propionate, diflorasone diacetate, fluocinonide, and halobetasol propionate. Certain topical corticosteroids are considered high potency such as, for example, amcinonide, desoximetasone, halcinonide, and triamcinolone acetonide. Certain topical corticosteroids are considered medium potency, such as, for example, betamethasone valerate, clocortolone pivalate, fluocinolone acetonide, flurandrenolide, fluocinonide, fluticasone propionate, hydrocortisone butyrate, hydrocortisone valerate, mometasone furoate, and prednicarbate. Certain topical corticosteroids are considered low potency, such as, for example, alclometasone dipropionate, desonide, and hydrocortisone. “Inhalable corticosteroid” means a corticosteroid that is suitable for delivery by inhalation. Exemplary inhalable corticosteroids are fluticasone, beclomethasone dipropionate, budenoside, mometasone furoate, ciclesonide, flunisolide, triamcinolone acetonide and any other corticosteroid currently available or becoming available in the future. Examples of corticosteroids that can be inhaled and are combined with a long-acting beta2-agonist include, but are not limited to: budesonide/formoterol and fluticasone/salmeterol.

The bacteria compositions described herein can be included in regulatory agency approved kits such as an FDA-approved kit.

According to a specific embodiment, the therapeutically effective amount of the bacteria causes a reduction in SCORAD score as compared to said SCOARD score of the subject prior to treatment with said bacteria, said reduction being maintained for at least 10 weeks (e.g., at least 12 weeks, at least 14 weeks, at least 18 weeks, at least 24 weeks, at least 26 weeks, at least 52 weeks) following first cycle treatment with said bacteria.

According to a specific embodiment, the therapeutically effective amount of the bacteria results in colonization of the bacteria in the intestines of the subject.

According to another aspect of the present invention there is provided a method of analyzing whether a bacterial treatment is effective for treating a subject having AD, the method comprising:

(a) providing the above-mentioned bacteria e.g., compositions, to the subject; and

(b) analyzing the amount of at least one bacterial species in the gut microbiome of the subject, said bacterial species being selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella. SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, wherein an increase in the amount of said bacterial species following said providing as compared to the amount of said bacterial species prior to said providing is indicative that the composition was effective at treating the subject.

Analyzing the amount of any of the above disclosed species may be effected by determining one or more DNA sequences. In some embodiments, one or more DNA sequences comprises any DNA sequence that can be used to differentiate between different microbial types. In certain embodiments, one or more DNA sequences comprises 16S rRNA gene sequences. In certain embodiments, one or more DNA sequences comprises 18S rRNA gene sequences. In some embodiments, 1, 2, 3, 4, 5, 10, 15, 20, 25, 50, 100, 1,000, 5,000 or more sequences are amplified.

16S and 18S rRNA gene sequences encode small subunit components of prokaryotic and eukaryotic ribosomes respectively. rRNA genes are particularly useful in distinguishing between types of bacterias because, although sequences of these genes differ between microbial species, the genes have highly conserved regions for primer binding. This specificity between conserved primer binding regions allows the rRNA genes of many different types of bacterias to be amplified with a single set of primers and then to be distinguished by amplified sequences.

In some embodiments, a microbiota sample (e.g. gut microbiome) is directly assayed for a level or set of levels of one or more DNA sequences. Methods of isolating microbial DNA are well known in the art. Examples include but are not limited to phenol-chloroform extraction and a wide variety of commercially available kits, including QIAamp DNA Stool Mini Kit (Qiagen, Valencia, Calif.).

In some embodiments, a level or set of levels of one or more DNA sequences is determined by amplifying DNA sequences using PCR (e.g., standard PCR, semi-quantitative, or quantitative PCR) and then sequencing. In some embodiments, a level or set of levels of one or more DNA sequences is determined by amplifying DNA sequences using quantitative PCR. These and other basic DNA amplification procedures are well known to practitioners in the art and are described in Ausebel et al. (Ausubel F M, Brent R, Kingston R E, Moore D, Seidman J G, Smith J A, Struhl K (eds). 1998. Current Protocols in Molecular Biology. Wiley: New York).

In some embodiments, DNA sequences are amplified using primers specific for one or more sequence that differentiate(s) individual microbial types from other, different microbial types. In some embodiments, 16S rRNA gene sequences or fragments thereof are amplified using primers specific for 16S rRNA gene sequences. In some embodiments, 18S DNA sequences are amplified using primers specific for 18S DNA sequences.

In some embodiments, a level or set of levels of one or more 16S rRNA gene sequences is determined using phylochip technology. Use of phylochips is well known in the art and is described in Hazen et al. (“Deep-sea oil plume enriches indigenous oil-degrading bacteria.” Science, 330, 204-208, 2010), the entirety of which is incorporated by reference. Briefly, 16S rRNA genes sequences are amplified and labeled from DNA extracted from a microbiota sample. Amplified DNA is then hybridized to an array containing probes for microbial 16S rRNA genes. Level of binding to each probe is then quantified providing a sample level of microbial type corresponding to 16S rRNA gene sequence probed. In some embodiments, phylochip analysis is performed by a commercial vendor. Examples include but are not limited to Second Genome Inc. (San Francisco, Calif.).

In some embodiments, determining a level or set of levels of one or more types of bacteria comprises determining a level or set of levels of one or more microbial RNA molecules (e.g., transcripts). Methods of quantifying levels of RNA transcripts are well known in the art and include but are not limited to northern analysis, semi-quantitative reverse transcriptase PCR, quantitative reverse transcriptase PCR, and microarray analysis.

Methods for sequence determination are generally known to the person skilled in the art. Preferred sequencing methods are next generation sequencing methods or parallel high throughput sequencing methods. For example, a bacterial genomic sequence may be obtained by using Massively Parallel Signature Sequencing (MPSS). An example of an envisaged sequence method is pyrosequencing, in particular 454 pyrosequencing, e.g. based on the Roche 454 Genome Sequencer. This method amplifies DNA inside water droplets in an oil solution with each droplet containing a single DNA template attached to a single primer-coated bead that then forms a clonal colony. Pyrosequencing uses luciferase to generate light for detection of the individual nucleotides added to the nascent DNA, and the combined data are used to generate sequence read-outs. Yet another envisaged example is Illumina or Solexa sequencing, e.g. by using the Illumina Genome Analyzer technology, which is based on reversible dye-terminators. DNA molecules are typically attached to primers on a slide and amplified so that local clonal colonies are formed. Subsequently one type of nucleotide at a time may be added, and non-incorporated nucleotides are washed away. Subsequently, images of the fluorescently labeled nucleotides may be taken and the dye is chemically removed from the DNA, allowing a next cycle. Yet another example is the use of Applied Biosystems' SOLiD technology, which employs sequencing by ligation. This method is based on the use of a pool of all possible oligonucleotides of a fixed length, which are labeled according to the sequenced position. Such oligonucleotides are annealed and ligated. Subsequently, the preferential ligation by DNA ligase for matching sequences typically results in a signal informative of the nucleotide at that position. Since the DNA is typically amplified by emulsion PCR, the resulting bead, each containing only copies of the same DNA molecule, can be deposited on a glass slide resulting in sequences of quantities and lengths comparable to Illumina sequencing. A further method is based on Helicos' Heliscope technology, wherein fragments are captured by polyT oligomers tethered to an array. At each sequencing cycle, polymerase and single fluorescently labeled nucleotides are added and the array is imaged. The fluorescent tag is subsequently removed and the cycle is repeated. Further examples of sequencing techniques encompassed within the methods of the present invention are sequencing by hybridization, sequencing by use of nanopores, microscopy-based sequencing techniques, microfluidic Sanger sequencing, or microchip-based sequencing methods.

According to one embodiment, the sequencing method allows for quantitating the amount of bacteria—e.g. by deep sequencing such as Illumina deep sequencing.

As used herein, the term “deep sequencing” refers to a sequencing method wherein the target sequence is read multiple times in the single test. A single deep sequencing run is composed of a multitude of sequencing reactions run on the same target sequence and each, generating independent sequence readout.

In some embodiments, determining a level or set of levels of one or more types of bacterias comprises determining a level or set of levels of one or more microbial polypeptides. Methods of quantifying polypeptide levels are well known in the art and include but are not limited to Western analysis and mass spectrometry.

As mentioned herein above, as well as (or instead of) analyzing the level of bacteria, the present invention also contemplates analyzing the level of microbial products.

Examples of microbial products include, but are not limited to mRNAs, polypeptides, carbohydrates and metabolites.

In some embodiments, the presence, level, and/or activity of metabolites of at least ten species of bacterias are measured. In other embodiments, the presence, level, and/or activity of metabolites of between 5 and 50 species of bacterias are measured. In other embodiments, the presence, level, and/or activity of metabolites of between 5 and 20 species of bacteria are measured. In other embodiments, the presence, level, and/or activity of metabolites of between 5 and 100 species of bacterias are measured. In some embodiments, the presence, level, and/or activity of metabolites of between 100 and 1000 or more species of bacterias are measured. In other embodiments, the presence, level, and/or activity of metabolites of all bacteria within the microbiome are analyzed. In other embodiments, the presence, level, and/or activity of metabolites of all bacterias within the microbiome are measured.

As mentioned, an increase in the amount of at least one, two, three, four, five or all of the of the disclosed species in the gut mirobiome sample following the transplantation as compared to the amount of the same species in the gut microbiome sample prior to the transplantation is indicative that the composition was effective at treating the subject. In one embodiment, the increase is at least 10% increase, 20% increase, 30% increase, 40% increase, 50% increase or greater.

The analysis may be taken 1 day, 2 days, 3 days, 4 days, five days, six days, 1 week, 2 weeks, 3 weeks, 4 weeks, more than 1 month, more than 2 months, more than 3 months, more than 5 months, more than 6 months following the transplant.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-IT Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Methods

Trial population. Eligible AD patients treated at the Dermatologic department at the Tel Aviv Medical Center. Patients included were >18 years of age, with moderate-to-severe atopic dermatitis, as defined by a Scoring Atopic Dermatitis Score (SCORAD) score >25, with disease duration minimum of 3 years that was inadequately controlled by topical and systemic therapy. Key exclusion criteria comprised of another concomitant active dermatologic disease, pregnancy, systemic therapy including phototherapy within 4 weeks before the beginning of the study.

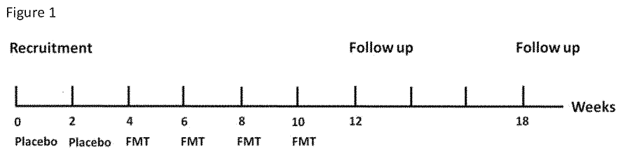

Study design and oversight. This was a prospective, single-blinded, placebo-controlled cross-over pilot study that was designed to assess the safety and clinical efficacy of FMT for the treatment of moderate and severe AD and to assess the change in the fecal microbiota following FMT in the study population. All patients were assessed for clinical AD activity at baseline, after which they had received 2 doses of placebo FMTs, followed by 4 treatment FMTs (from healthy stool donors) each 2 weeks apart (FIG. 1). Each patient received FMTs from a single donor (one of 3 available stool donors). However, if stool donations from a certain donor were no longer available, stool donor was changed. The clinical activity of AD, adverse events and the fecal microbiome profile were evaluated at the beginning of the study, before every FMT, and 1-8 months after the last FMT, using the SCORAD score.

During the study period, patients were allowed to use only topical therapy including emollients and glucocorticoids or calcineurin inhibitors.

The protocol was approved by the ethics review board of the Tel Aviv Medical Center. All patients provided written informed consent in accordance with the principles of the Declaration of Helsinki. There was no commercial support for the trial. All the inventors vouch for the accuracy and completeness of the data, for the adherence of the trial to the protocol, and for the reporting of adverse events.

FMT preparation and delivery. Volunteer donors were healthy, non-pregnant adults aged 18 to 50 years, with a normal body mass index of 18.5-24.9 kg/m². They were excluded for any significant medical history or for any use of antibiotics in the preceding 3 months. Candidates were eligible according to the Israeli Ministry of Health guidelines which include a physical examination and elaborative laboratory screening tests (not shown), including fecal enteric pathogens (through cultures, and PAN-PCR testing (Biofire Diagnostics, gastrointestinal panel)), serum antibodies to hepatitis A, B, and C; human immunodeficiency virus; HTLV, and Treponema pallidum as well as celiac, CBC (and additional tests that comply with the guidelines of the Israeli Ministry of Health). Three healthy volunteers that underwent the donor screening procedure served as stool donors for the current study. Stool was delivered within minutes post defecation in a clean closed plastic container and was processed at the Tel Aviv Medical Center stool bank facility to prepare capsulized FMT. The preparation of capsulized FMT was performed similar to the procedure previously described¹². Briefly, fecal material was diluted with normal saline (600 ml/100 g of fecal material), filtered and concentrated in a centrifuge. The pellet was suspended in sterile saline and glycerol (20%) that was added as a bacterial cryoprotectant. This material was then pipetted into acid-resistant capsules (DRCaps, Capsugel), which were closed and then secondarily sealed with additional set of capsules. Capsules were stored frozen at −80° C. Placebo capsules were identical visually, and contained diluted glycerol only.

Capsulized FMT procedure: FMT was administered two doses of 15 FMT capsules on two consecutive days (a total of 30 capsules), at the Bacteriotherapy clinic of the TLVMC. On the day of administration, capsules frozen at −80° C. were taken out of the freezer and transported to the clinic on ice. Fifteen capsules were handed individually to the patient and the patient orally ingested the capsules immediately with some water. Patients were asked to fast overnight prior to capsule intake.

Efficacy end points. The study primary end point was the overall mean change from baseline of the SCORAD score, described and validated by the European Task Force on Atopic Dermatitis, used for the assessment of the severity of AD^{13,14}. The secondary endpoint was safety of FMT for AD patients and the relation of clinical improvement to the change in the fecal microbial species 2 weeks after each FMT, and 8 weeks or more after the last FMT.

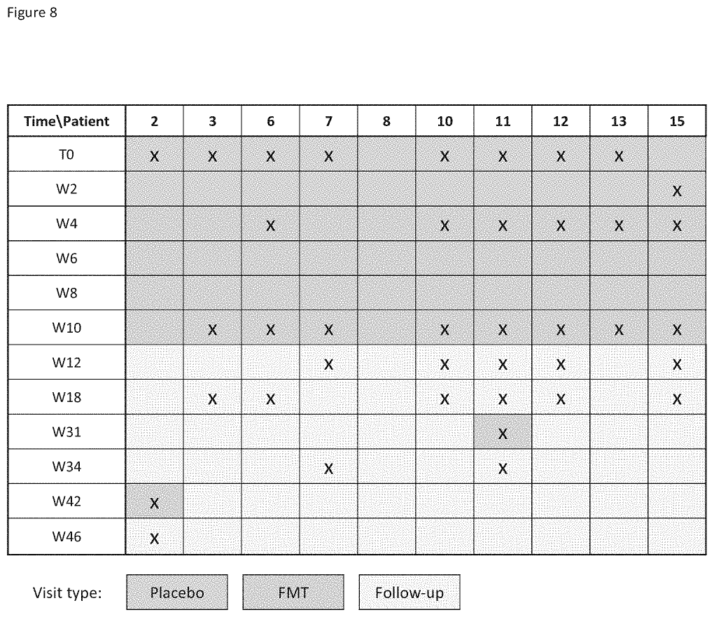

Fecal microbial analysis. In order to examine whether the clinical effect may be mediated by colonization of new bacterial strains, a robust and sensitive method was developed to calculate pairwise DNA sequence dissimilarity between bacterial strains of the same species across distinct metagenomics samples (not shown). Donor stool samples that were used for FMT capsules and stool samples that were collected from the patients during the study period (FIG. 8) were sequenced into metagenomics reads using Illumina NextSeq. Reads were mapped to reference genomes based on the ‘iterative coverage-based read assignment’ (ICRA) algorithm¹⁵. Reads that were mapped by ICRA were piled up to obtain per-position variant information for every detected species. Difference in the variant of a particular species at a given position between two samples was defined as having no intersection between the set of detected alleles in the two samples being compared. The estimated species DNA sequence dissimilarity for a pair of samples is then the number of different positions divided by the total number of positions being compared (CP):

$$dissimilarity ( S , x , y , d ) = \frac{1}{\vert CP \vert} \sum d ( x [ i ] , y [ i ] )$$

for species S, samples x, y with minimum of 20 kbp CP, and a comparison method d.

The CP cut-off means that dissimilarities below 0.00005 (=1/20000) changes per base pair can differ due to a different number of CP. We therefore define the minimal dissimilarity detection threshold to be 0.00005 changes per base pair.

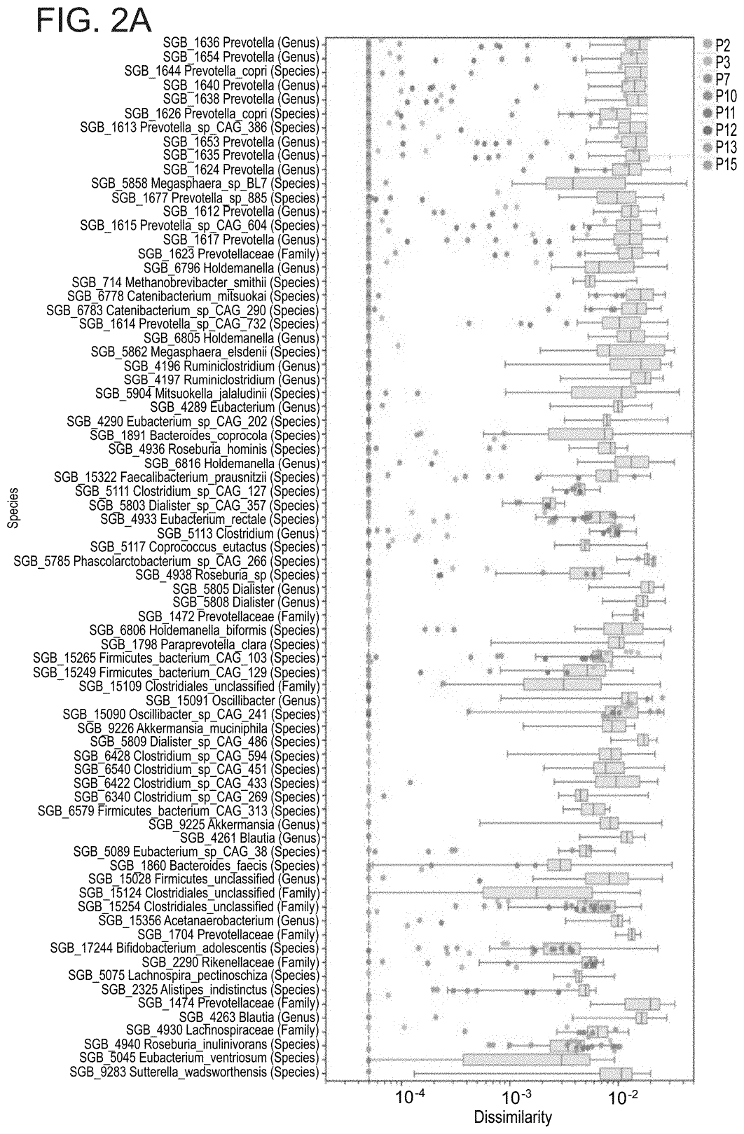

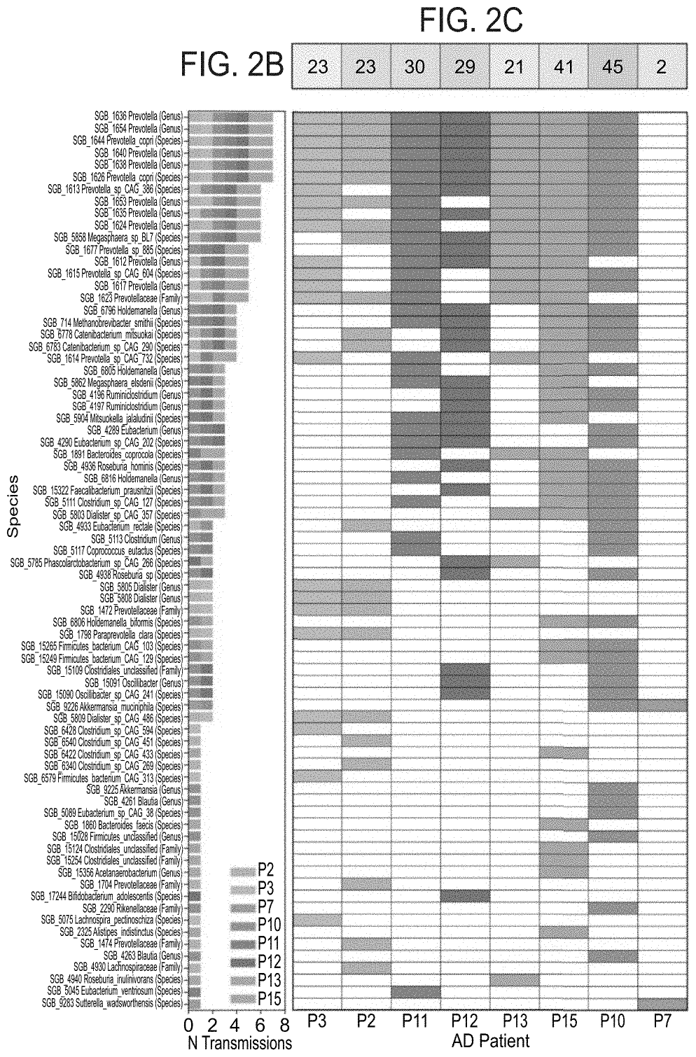

Comparison of patient-donor dissimilarities across different study time points to intra-host dissimilarities in a healthy reference population^{16,17} showed unusually low (below the detection threshold of 5e-5) dissimilarities between patient and his/her donor after an FMT was performed, indicative of transmission and colonization of donor strains in the patient (FIG. 2A). Thus, dissimilarities below the defined detection threshold are referred to as “transmission events” or “strain sharing”. An aggregated dissimilarity score was defined for pair of samples as:

$${sample}_{dissimilarity} ( X , Y ) = 1 - \frac{N}{N + 1}$$

where N is the number of shared strains between samples X and Y.

Statistical analysis. Significance levels were calculated by using The Wilcoxon signed-rank test for comparison between two matched samples. Correlation between SCORAD reduction and sample-dissimilarity was calculated according to Pearson correlation method. Both were implemented by using scipy.stats python package.

Sample processing. Donor stool samples that were used for FMT capsules and stool samples that were collected from the patients during the study period were sequenced into metagenomics reads using Illumina NextSeq. Illumina adapters, low quality reads and human associated reads was filtered out. Reads were mapped to reference genomes, that were processed from EMBL proGenomes database, based on the ‘iterative coverage-based read assignment’ (ICRA) algorithm as described in Zeevi, et al.². Reads with ICRA genome assignment probability of less than 0.99 was filtered out.

Reads that were mapped by ICRA were piled up to obtain per-position variant information. This step resulted in a Species Allele Counts Matrix (SACM) for each species that was detected in the sample. In every SACM only positions with at least 5× read coverage was retained. Positions with lower coverage were removed from further analysis and are referred to as uncovered positions. The following steps were performed only on SACMs with 30% or more covered positions (read coverage >=1×).

### Reference Cohort

The following was used as healthy reference population:

TABLE 1

| Inner | | | No. Of | No. of | | |
|---|---|---|---|---|---|---|
| Name | Reference | Accessions | Samples | individuals | Country | Short description |
| PNP³ | Zeevi, David, | PRJEB11532 | 1730 | 999 | Israel | Israeli adult individuals as |
| | et al. 2015 | | | | | part of the |
| | | | | | | personalized nutrition |
| | | | | | | project. |
| HMP⁴ | Lloyd-rice, | PRJNA48479, | 474 | 228 | USA | US American adult |
| | Jason, et al. | PRJNA275349 | | | | individuals as part of |
| | 2017 | | | | | the human microbiome |
| | | | | | | consortium. |

Healthy reference population analysis. The healthy reference population data was generated by calculating inter-cohort pairwise dissimilarities. In order to prevent bias of the results towards individuals that have more than one sample, for every species and a pair of individuals only one representative comparison was retained. If more than one comparison was available, comparison that maximizes the number of reads that were assigned to the corresponding SACMs was chosen.

Dissimilarity to baseline, post-placebo and donors' samples In many cases is was possible to calculate species dissimilarity between the patient's samples and multiple baseline/post-placebo/donor's samples. In these cases, to avoid bias, and to simplify the analysis, only the lowest dissimilarity was retained for further analysis.

### Example 2

### Reduction in SCORAD in FMT-Treated AD Patients

A total of 15 AD patients were enrolled to the trial. Four did not enter the study due to inability to comply with the trial regimen, and one patient decided to start an already approved medication due to disease exacerbation during the first two weeks of the trial (placebo period). Another patient (patient 6) breached the study protocol by using enemas, thus, possibly effecting the gut microbiome composition. Although he had clinical improvement he was not included in the clinical and fecal microbiome analysis. Nine patients (5 males and 4 females), average age of 44.6 years (range 24 to 68 years), were finally enrolled and completed the study. One of these patients (patient 8) failed to deliver stool samples and is therefore not included in the fecal microbiome analysis.

No adverse events were reported during the study. Prior to treatment, the average SCORAD score of all 9 patients eligible for analysis was 51.2±16.2, with 4 patients classified as moderate disease (SCORAD 25-50), and 5 as severe (SCORADE >50). Prior to enrolment, all patients received stable topical therapy as well as one or more systemic treatment (Table 2); 6 patients received phototherapy, 2 were treated with cyclosporine, 5 patients received oral prednisone, one patient was treated with methotrexate and the loading dose of Dupilumab induction 6 weeks prior to commencing the trial.

TABLE 2
Patients' characteristics and their SCORAD scores during the study period.

| Patient # | | 2 | 3 | 6 | 7 | 8 | 10 | 11 | 12 | 13 | 15 |
|---|---|---|---|---|---|---|---|---|---|---|---|
| Age (yrs) | | 41 | 56 | 50 | 68 | 45 | 35 | 59 | 24 | 37 | 37 |
| Gender | | f | m | m | f | f | m | m | f | m | m |
| Previous | | Phot | Photo | Cyclo- | Photo, | Cyclo- | Prednisone | Photo, | Prednisone | Cyclo- | Photo, |
| systemic | | | | sporine, | prednisone | sporine, | | Metho- | | sporine, | Prednisone |
| therapy | | | | Predni | | Photo | | trexate, | | Prednisone, | |
| | | | | | | | | Dupilu | | | |
| | | | | | | | | [symbol] | | | |
| Time | Intervention | | | | | | | | | | |
| point | | | | | | | | | | | |
| (week) | | | | | | | | | | | |
| 0 | Placebo | 70 | 41.5 | 64 | 70 | 31 | 27.5 | 41.5 | 61 | 57.5 | 61 |
| | 1 | | | | | | | | | | |
| 2 | Placebo | | | 67 | 38 | 24.5 | | | 58 | | 58 |
| | 2 | | | | | | | | | | |
| 4 | FMT1 | 67 | 39.5 | 25 | 48.5 | 32 | 27 | 62.5 | 71 | 60 | 54.5 |
| 6 | FMT2 | 41 | 18 | 15 | 4 | | 14.5 | 29.5 | 15 | 28 | 49 |
| 8 | FMT3 | 25.5 | 12 | 11.5 | 4 | 7 | 9 | 17.5 | 22 | 44 | 38 |
| 10 | FMT4 | 33.5 | 10.5 | 9 | 4 | 14 | 11 | 19 | 29 | 22 | 39 |
| 12 | | 31 | 6 | 9 | 0 | 40 | 11 | 10.5 | 30 | 11 | 34 |
| 18 | | 11 | | | | | 4.5 | 8 | | | 14 |
| 21 | | 11 | | 10 | | | | | | | |
| 22 | | | | | 0 | | | | | | |
| 23 | | | | | | | 5 | | | | |
| 25 | | 11 | | | | | | | | | |
| 26 | | | | 10 | | | | | | | |
| 29 | | 10 | | | | | | | | | |
| 31 | FMT5 | | | | | | | 36.5 | | | |
| | (patient | | | | | | | | | | |
| | #11) | | | | | | | | | | |
| 32 | | | 5 | | | | | | | | |
| 33 | | | | | 5 | | | | | | |
| 34 | | | | | | | | 22 | | | |
| 42 | FMT5 | 49 | | | | | | | | | |
| | (patient | | | | | | | | | | |
| | #2) | | | | | | | | | | |
| 46 | | 10 | | | | | | | | | |

Abbreviations:
FMT— fecal microbial transplantation;
yrs— years;
f— female;
m— male [symbol]
indicates data missing or illegible when filed

At the end of the placebo part of the trial (end of week 4), there was no significant change in AD severity (SCORAD augmentation of 2.5%±22.1%, Wilcoxon P>0.85). One patient exhibited improvement of 31%, four patients exhibited improvement in the range 1-11%, and the remaining four patients experienced worsening of the disease by an average SCORAD elevation of 19% (range 3-51%).

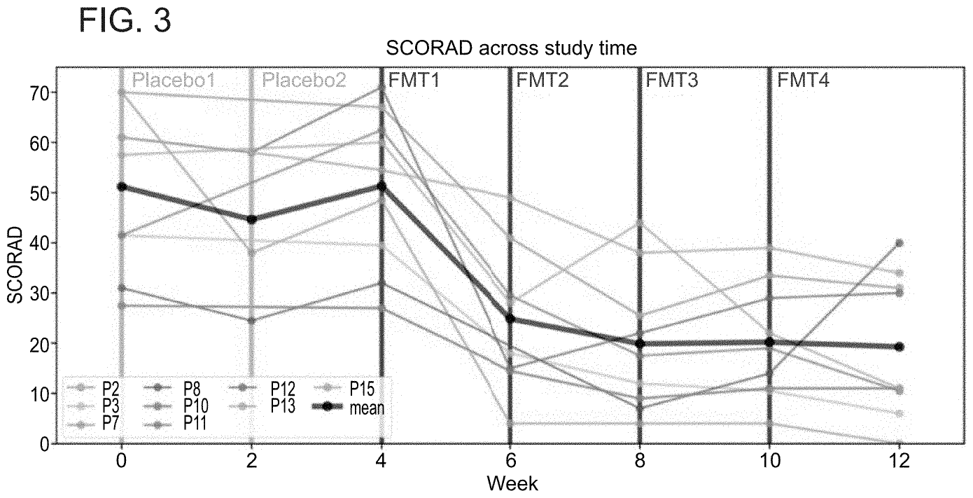

All 9 patients who started the study completed the FMTs protocol. Disease severity, assessed by the SCORAD score, quickly improved in all patients (FIG. 3). Following each FMT there was a significant reduction in the average SCORAD score compared to the score at week 4, the end of the placebo period and beginning of the FMT treatment period (53.3%±23.1%, 62.9%±20%, 61.2%±16.3%, and 59.2%±34.9% respectively). The maximal SCORAD reduction during the entire FMT's and follow-up period, which lasted in average 18 weeks (range 2-36 weeks), was even higher, with an average of 84% (Table 2 above).

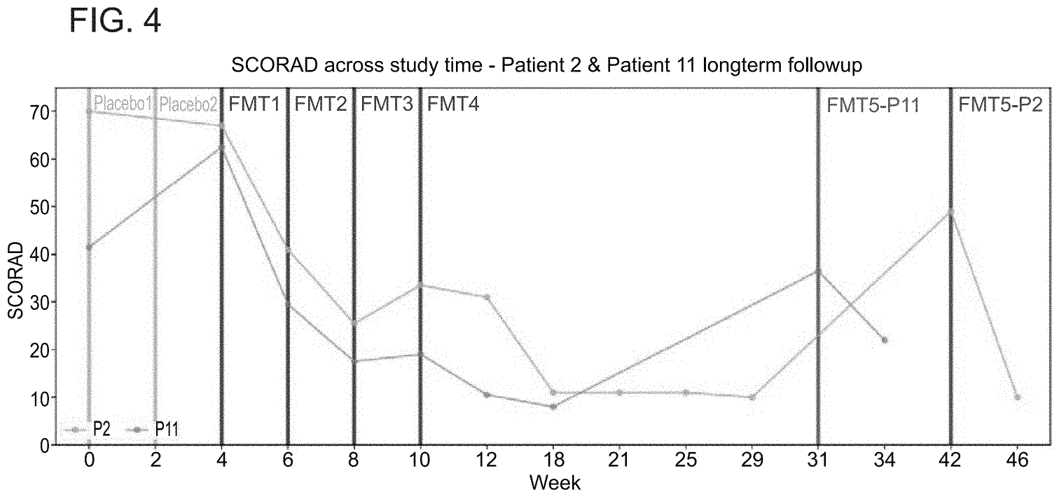

Five patients maintained the response during their follow-up period lasting 14 weeks in average (range 8-22 weeks). Four patients had a relapse immediately after the FMTs or during the follow-up period. Two patients (Patients No 2 and 11) that had a maximal SCORAD reduction of 85% and 87% had an exacerbation, reflected by an increase of the score from 11 to 49, and from 8 to 36.5, 32 and 21 weeks after the last FMT, respectively. Nevertheless, despite the exacerbation, they still had a SCORAD reduction of 26.9% and 41.6% from baseline. Both patients received an additional single FMT and experienced disease improvement with a decrease of the SCORAD score of 80% and 40% respectively (FIG. 4).

Other two participants with a good initial response comprising of SCORAD reduction of 78% and 79% had a quick relapse, with an elevation of the SCORAD score. The first (patient No 8) at week 12, from 7 to 40, reflecting worsening even from the initial score of 32, and the second (patient No 12) at week 10, from 22 to 29, still better than the baseline by 59%. The exacerbation led to a switch to different treatment.

Although 3 different healthy stool donors participated in the study, there was no significant difference in terms of response rate among the recipients. It should be noted that most patients received all FMTs from one donor, however, due to lack of available stool donations at certain time points, three patients received FMTs from two donors (Table 2 above).

### Example 3

### Donor to Patient Strain Transmission and Clinical Effect

A strain dissimilarity score was developed between two species from two different samples, based on comparing the basepair similarity across their genomes in the two samples being compared (Methods). To examine whether this method can detect strain transmission, patient-donor dissimilarities across different study time points were calculated as these may have transmission events. Patient-donor dissimilarity scores were compared to inter-host dissimilarity scores that were computed from a large reference population of more than 1200 healthy subjects^{16,17}. It was found that many (138 of 270 comparisons, 51%) cases of unusually low dissimilarity (below the detection threshold of 0.00005) between patients and their donors after an FMT. Such low dissimilarity scores were rarely found when comparing inter-host dissimilarities in study healthy population (5775 of 4.6M comparisons, 0.13%, FIG. 2A) and also when comparing patients to their donors at either baseline (0 of 57 comparisons, 0%) or after two doses of placebo (0 of 56 comparisons, 0%). These results suggest that dissimilarity scores below the detection threshold are indicative of transmission events and colonization of donor strains in the patients and thus dissimilarities below the defined detection threshold are referred to as “transmission events” or “strain sharing”.

Overall, 48 transmission events were detected from donors to patients, with an average of 6±3.4 transmission events per donor, and at least one transmission event was found in the 8 allegeable patients for whom stool samples were available (FIG. 2B). Thirteen species were transmitted into more than one patient, with Prevotella copri standing out as it was transmitted in 7 of the 8 patients (FIG. 2C).

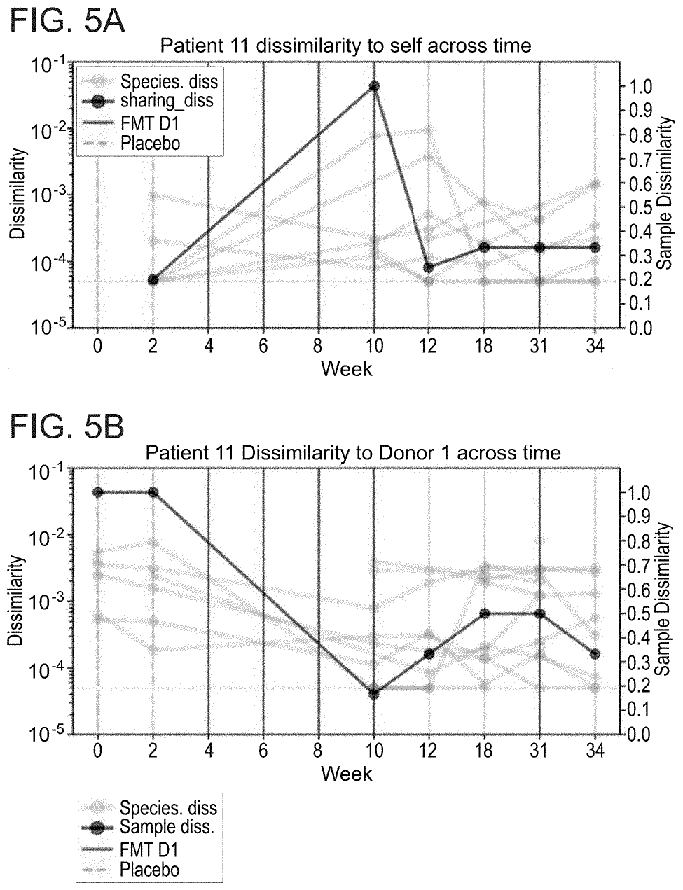

Next samples of the same patient were compared to themselves to study how the gut microbiome changes throughout the study from the point of view of each individual patient (FIG. 3 and not shown). For most patients, a high similarity was found between strains at baseline and after placebo treatment, indicative of high microbiome stability. In contrast, following the FMTs, high dissimilarities to strains at baseline were found, indicative of transmission events and changes in the microbiome strain pool. As expected, when comparing patients to their donors, a mirror picture was revealed, whereby there was high dissimilarity between the donor and both baseline and post-placebo samples of each patient, and low dissimilarity after the FMT. Taken together, these results suggest that without any treatment (including placebo) bacterial strains of patients are stable over time, but following FMT some strains are transmitted from donors and replace previous strain that exist in patients (FIG. 5B).

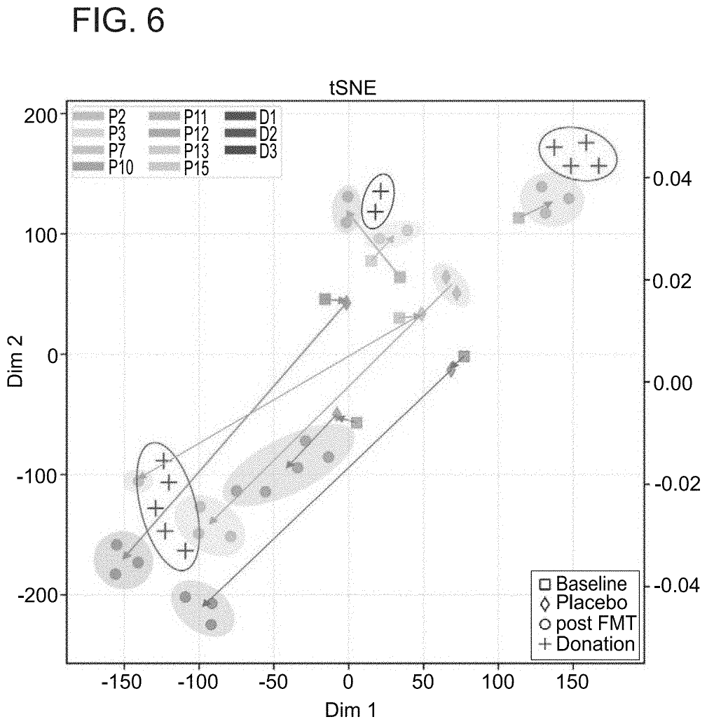

As a more global view of the results, an aggregated dissimilarity score was devised between two samples based on the dissimilarity scores of all species that could be compared between the samples (Methods). t-distributed stochastic neighbor embedding (t-SNE) analysis was performed on the pairwise dissimilarity matrix of all samples (FIG. 6). This global view demonstrates that baseline and placebo samples are randomly distributed across the space while post-FMT samples of patients' cluster near their donor samples. Thus, following FMT treatment, strains in the microbiome composition of patients becomes similar to those of their donors, suggestive of bacterial transmission events that result in large shifts in the composition.

A similar analysis was performed on a Bray-Curtis dissimilarity matrix that was computed based on the relative abundance data of the samples (not shown). In this case, most of the patients' post-FMT clusters are randomly distributed across the space and are not located near their donor, suggesting that the effect of the FMTs is more reflected by specific strains transmission than by changes in the relative abundance.

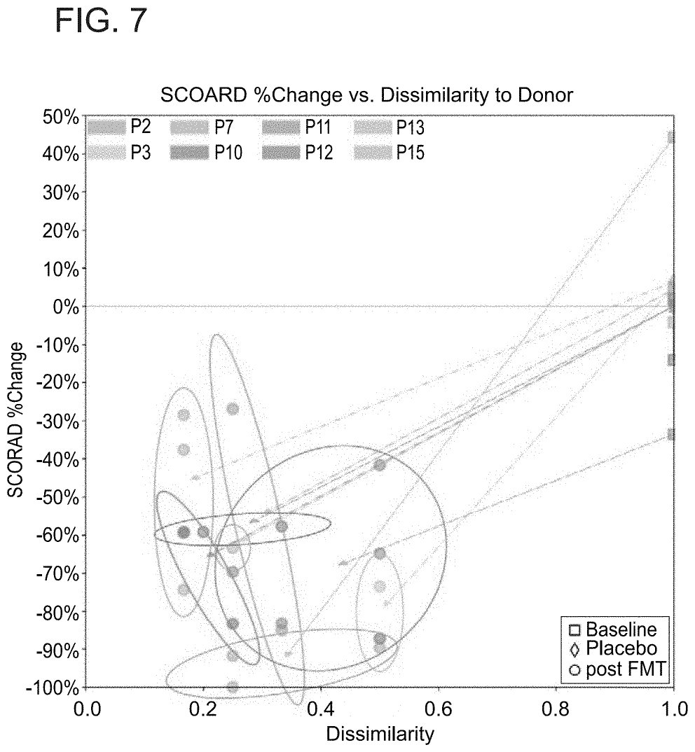

Finally, we found a significant correlation between the improvement in disease severity (change in the SCORAD score) and the sample dissimilarity between donor and their patients (R=0.76, P<4*10-8, FIG. 7). These results demonstrate an association between the degree of strain transmission from donor to patient and clinical improvements, suggestive of a possible casual effect of bacterial strain transmission from donor to patient in treatment of AD.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Kowalska-Ol [symbol] dzka E, Czarnecka M, Baran A. Epidemiology of atopic dermatitis in Europe. J Drug Assess 2019; 8(1):126-8.
2. Renert-Yuval Y, Guttman-Yassky E. What's new in atopic dermatitis. Dermatol Clin 2019; 37(2):205-13.
3. Wollenberg A, Barbarot S, Bieber T, et al. Consensus-based European guidelines for treatment of atopic eczema (atopic dermatitis) in adults and children: part I. J Eur Acad Dermatol Venereol 2018; 32(5):657-82.
4. Huang J T, Abrams M, Tlougan B, Rademaker A, Paller A S. Treatment of Staphylococcus aureus colonization in atopic dermatitis decreases disease severity. Pediatrics 2009; 123(5):e808-14.
5. Pothmann A, Illing T, Wiegand C, Hartmann A A, Elsner P. The microbiome and atopic dermatitis: A review. Am J Clin Dermatol 2019;
6. Szántó M, Dózsa A, Antal D, Szabó K, Kemény L, Bai P. Targeting the gut-skin axis-Probiotics as new tools for skin disorder management? Exp Dermatol 2019;
7. Pan H-H, Lue K-H, Sun H-L, Ku M-S. Gastroenteritis during infancy is a novel risk factor for allergic disease. Medicine 2019; 98(35):e16540.
8. Foliaki S, Pearce N, Björkstén B, et al. Antibiotic use in infancy and symptoms of asthma, rhinoconjunctivitis, and eczema in children 6 and 7 years old: International Study of Asthma and Allergies in Childhood Phase III. J Allergy Clin Immunol 2009; 124(5):982-9.
9. Reddel S, Del Chierico F, Quagliariello A, et al. Gut microbiota profile in children affected by atopic dermatitis and evaluation of intestinal persistence of a probiotic mixture. Sci Rep 2019; 9(1):4996.
10. Petersen E B M, Skov L, Thyssen J P, Jensen P. Role of the gut microbiota in atopic dermatitis: A systematic review. Acta Derm Venereol 2019; 99(1):5-11.
11. Lee J, Seto D, Bielory L. Meta-analysis of clinical trials of probiotics for prevention and treatment of pediatric atopic dermatitis. J Allergy Clin Immunol 2008; 121(1):116-121.e11.

What is claimed is:

1. A method of treating atopic dermatitis (AD) in a human subject in need thereof comprising administering to the subject a therapeutically effective amount of at least two species of bacteria, wherein said at least two species of said bacteria are selected from the group consisting of
SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, thereby treating the atopic dermatitis, wherein said bacteria are not comprised in a faecal microbiota transplantation (FMT).

2. A method of treating atopic dermatitis (AD) in a human subject in need thereof comprising administering to the subject a therapeutically effective amount of between two and 100 species of bacteria, wherein at least two of said species are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB_9283 Sutterella_wadsworthensis, thereby treating the atopic dermatitis.

3. A composition comprising a therapeutically effective amount of between two and 100 species of bacteria, wherein at least two of said species are selected from the group consisting of SGB_1636 Prevotella, SGB_1654 Prevotella, SGB_1644 Prevotella_copri, SGB_1640 Prevotella, SGB_1638 Prevotella, SGB_1626 Prevotella_copri, SGB_1613 Prevotella_sp_CAG_386, SGB_1653 Prevotella, SGB_1635 Prevotella, SGB_1624 Prevotella, SGB_5858 Megasphaera_sp_BL7, SGB_1677 Prevotella_sp_885, SGB_1612 Prevotella, SGB_1615 Prevotella_sp_CAG_604, SGB_1617 Prevotella, SGB_1623 Prevotellaceae, SGB_6796 Holdemanella, SGB_714 Methanobrevibacter_smithii, SGB_6778 Catenibacterium_mitsuokai, SGB_6783 Catenibacterium_sp_CAG_290, SGB_1614 Prevotella_sp_CAG_732, SGB_6805 Holdemanella, SGB_5862 Megasphaera_elsdenii, SGB_4196 Ruminiclostridium, SGB_4197 Ruminiclostridium, SGB_5904 Mitsuokella_jalaludinii, SGB_4289 Eubacterium, SGB_4290 Eubacterium_sp_CAG_202, SGB_1891 Bacteroides_coprocola, SGB_4936 Roseburia_hominis, SGB_6816 Holdemanella, SGB_15322 Faecalibacterium_prausnitzii, SGB_5111 Clostridium_sp_CAG_127, SGB_5803 Dialister_sp_CAG_357, SGB_4933 Eubacterium_rectale, SGB_5113 Clostridium, SGB_5117 Coprococcus_eutactus, SGB_5785 Phascolarctobacterium_sp_CAG_266, SGB_4938 Roseburia_sp, SGB_5805 Dialister, SGB_5808 Dialister, SGB_1472 Prevotellaceae, SGB_6806 Holdemanella_biformis, SGB_1798 Paraprevotella_clara, SGB_15265 Firmicutes_bacterium_CAG_103, SGB_15249 Firmicutes_bacterium_CAG_129, SGB_15109 Clostridiales_unclassified, SGB_15091 Oscillibacter, SGB_15090 Oscillibacter_sp_CAG_241, SGB_9226 Akkermansia_muciniphila, SGB_5809 Dialister_sp_CAG_486, SGB_6428 Clostridium_sp_CAG_594, SGB_6540 Clostridium_sp_CAG_451, SGB_6422 Clostridium_sp_CAG_433, SGB_6340 Clostridium_sp_CAG_269, SGB_6579 Firmicutes_bacterium_CAG_313, SGB_9225 Akkermansia, SGB_4261 Blautia, SGB_5089 Eubacterium_sp_CAG_38, SGB_1860 Bacteroides_faecis, SGB_15028 Firmicutes_unclassified, SGB_15124 Clostridiales_unclassified, SGB_15254 Clostridiales_unclassified, SGB_15356 Acetanaerobacterium, SGB_1704 Prevotellaceae, SGB_17244 Bifidobacterium_adolescentis, SGB_2290 Rikenellaceae, SGB_5075 Lachnospira_pectinoschiza, SGB_2325 Alistipes_indistinctus, SGB_1474 Prevotellaceae, SGB_4263 Blautia, SGB_4930 Lachnospiraceae, SGB_4940 Roseburia_inulinivorans, SGB_5045 Eubacterium_ventriosum and SGB 9283 Sutterella wadsworthensis.

4. The method of claim 1, wherein said bacteria are of the genus Prevotella, Holdemanella and/or Megasphaera.

5. The method of claim 1, wherein said bacteria are comprised in a well-defined preparation.

6. The method of claim 1, comprising at least one of SGB_1636 Prevotella, SGB_1654 Prevotella, SGB_1644 Prevotella_copri, SGB_1640 Prevotella, SGB_1638 Prevotella, SGB_1626 Prevotella_copri;
SGB_1636 Prevotella, SGB_1654 Prevotella, SGB_1644 Prevotella_copri, SGB_1640 Prevotella, SGB_1638 Prevotella, SGB_1626 Prevotella_copri, SGB_1653 Prevotella; or
SGB_1636 Prevotella, SGB_1654 Prevotella, SGB_1644 Prevotella_copri, SGB_1640 Prevotella, SGB_1638 Prevotella, SGB_1626 Prevotella_copri, SGB_1624 Prevotella.

7. The composition of claim 3, wherein less than 10% of the bacteria in the composition are of the genus Clostridium.

8. The composition of claim 3, wherein less than 10% of the bacteria in the composition are of the species Clostridium absonum, Clostridium argentinense, Clostridium baratii, Clostridium bifermentans, Clostridium botulinum, Clostridium butyricum, Clostridium cadaveris, Clostridium camis, Clostridium celatum, Clostridium chauvoei, Clostridium clostridioforme, Clostridium cochlearium, Clostridium difficile, Clostridium fallax, Clostridium felsineum, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium limosum, Clostridium malenominatum, Clostridium novyi, Clostridium oroticum, Clostridium paraputrificum, Clostridium perfringens, Clostridium piliforme, Clostridium putrefaciens, Clostridium putrificum, Clostridium ramosum, Clostridium sardiniense, Clostridium sartagoforme, Clostridium scindens, Clostridium septicum, Clostridium sordellii, Clostridium sphenoides, Clostridium spiroforme, Clostridium sporogenes, Clostridium subterminale, Clostridium symbiosum, Clostridium tertium, Clostridium tetani, Clostridium welchii, and/or Clostridium villosum.

9. The method of claim 1, wherein said administering comprises oral administration.

10. The method of claim 1, wherein said administering comprises topical administration.

11. The method of claim 1, wherein said bacteria are formulated for topical administration.

12. The method of claim 1, wherein the bacteria are lyophilized or spray-dried.

13. The method of claim 1, wherein said subject is an adult subject.

14. The method of claim 1, wherein said subject is 3-18 years of age.

15. The method of claim 1, wherein said subject exhibits moderate-to-severe AD with SCORAD score of >25.

16. The method of claim 1, wherein said therapeutically effective amount of the bacteria causes at least 40% reduction in SCORAD score as compared to said SCOARD score of the subject prior to treatment with said bacteria.

17. The method of claim 1, wherein said therapeutically effective amount of the bacteria causes a reduction in SCORAD score as compared to said SCOARD score of the subject prior to treatment with said bacteria, said reduction being maintained for at least 10 weeks following first cycle treatment with said bacteria.

18. The method of claim 1, wherein said therapeutically effective amount of the bacteria results in colonization of the bacteria in the intestines of the subject.

19. The method of claim 1, further comprising treating the subject with an anti AD therapy other than the bacteria.
